(19) United States
(12) Patent Application Publication
(10) Pub. No.: US 2026/0345575 A1
(43) Pub. Date: Nov. 5, 2026

(54) NOVEL NON-ORAL FORMULATIONS OF FACTOR XA INHIBITORS AND PROCESS FOR THEIR PREPARATION

(71) Applicant: Jodas Expoim Pvt. Ltd., Hyderabad (IN)

(72) Inventors: Tathagata DUTTA, Murshidabad (IN); Himanshu AGRAWAL, Indore (IN); Manisha MULCHANDANI, Barwani (IN); Jyoti LOOMBA, Hyderabad (IN)

(73) Assignee: Jodas Expoim Pvt. Ltd., Hyderabad (IN)

(21) Appl. No.: 19/103,856

(22) PCT Filed: Dec. 27, 2022
(86) PCT No.: PCT/IB2022/062795

§ 371 Date: Feb. 14, 2025

(30) Foreign Application Priority Data
Nov. 4, 2022 (IN) 202241063008

(51) Int. Cl. A61K 31/4545; A61K 31/44; A61K 31/444; A61K 31/519; A61K 31/5377; A61K 47/10; A61K 47/18; A61K 47/34

(52) U.S. Cl. CPC A61K 31/4545; A61K 31/44; A61K 31/444; A61K 31/519; A61K 31/5377; A61K 47/10; A61K 47/18; A61K 47/34

(57) ABSTRACT

A non-oral formulation of factor Xa inhibitors comprising factor Xa inhibitor and pharmaceutically acceptable excipients selected from vehicles, surface active agents, co-solvents and stabilizers. The present invention also relates to a process for preparing novel formulation of factor Xa inhibitor for non-oral route of administration.

### FIELD OF INVENTION

The present invention relates to a novel non-oral formulation of factor Xa inhibitors comprising factor Xa inhibitor and pharmaceutically acceptable excipients selected from vehicles, surface active agents, co-solvents and stabilizers.

The present invention also relates to a process for preparing novel formulation of factor Xa inhibitor for non-oral route of administration.

The present invention also relates to a novel non-oral formulation of factor Xa inhibitors for parenteral, topical, nasal, transdermal and ophthalmic administration.

### BACKGROUND OF INVENTION

Factor Xa inhibitors are small molecules that selectively and reversibly bind to the active site of activated factor X (Xa), which blocks the interaction with its substrate in a rapid and competitive fashion, therefore inhibiting the final effects of thrombin generation.

Several factor Xa inhibitors, such as Apixaban, Razaxaban, Betrixaban Rivaroxaban, and Edoxaban.

Apixaban is a known compound having the below structure and molecular weight 459.50 g/mol.

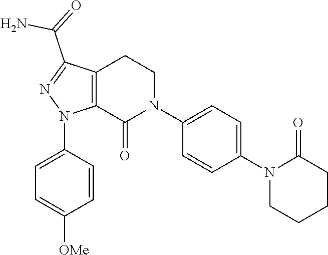 [SMILES: COc1ccc(-n2nc(C(N)=O)c3c2C(=O)N(c2ccc(N4CCCCC4=O)cc2)CC3)cc1]

Razaxaban is a known compound having the below structure and molecular weight 528.5 g/mol.

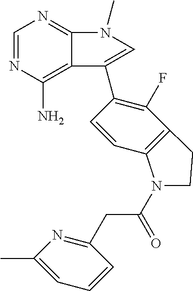 [SMILES: Cc1cccc(CC(=O)N2CCc3c2ccc(-c2cn(C)c4ncnc(N)c24)c3F)n1]

Edoxaban is a known compound having the below structure and molecular weight 548.1 g/mol.

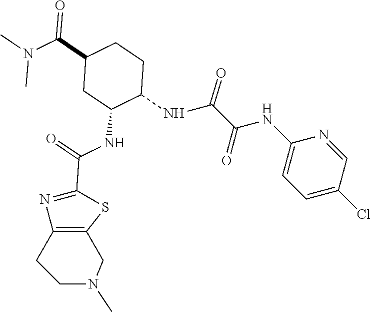 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

Rivaroxaban is a known compound having the below structure and molecular weight 435.9 g/mol.

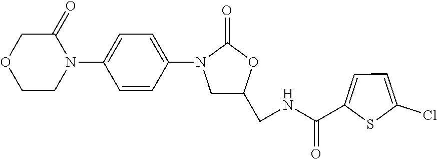 [SMILES: O=C(NCC1CN(c2ccc(N3CCOCC3=O)cc2)C(=O)O1)c1ccc(Cl)s1]

Betrixaban is a known compound having the below structure and molecular weight 451.9 g/mol.

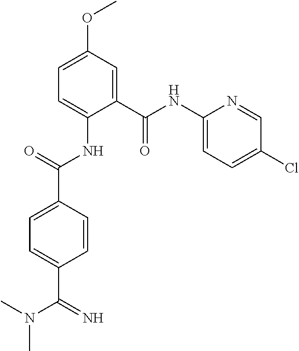 [SMILES: COc1ccc(NC(=O)c2ccc(C(=N)N(C)C)cc2)c(C(=O)Nc2ccc(Cl)cn2)c1]

U.S. patent application 2015/0224053 A1 teaches a oral liquid formulation comprising Apixaban and a vehicle, the vehicle comprising water and at least two solubilizers selected from the group consisting of a non-ionic surfactant, an ionic surfactant, a hydrophilic polymer, ethanol, a polyhydric alcohol, a polyethylene glycol, and a carbohydrate, wherein a solubility of Apixaban in the vehicle is at least 0.50 mg/mL.

PCT application WO 2007/022165 A2 teaches a pharmaceutical formulation comprising a factor Xa inhibitor and a substituted-β-cyclodextrin as solubilizing agent wherein the formulation is in the form of an injectable formulation.

Non-oral formulation of factor Xa inhibitor such as Apixaban or Razaxaban or Edoxaban or Rivaroxaban or Betrixaban eases the administration in subconscious, non-cooperative or pediatric or geriatric patients who have difficulty in swallowing a dosage form through oral route.

Factor Xa inhibitors such as Apixaban or Razaxaban are weak base. Reportedly, Apixaban has pH dependent instability and its free base form has extremely low aqueous solubility which is less than 0.04 mg/mL. Similarly, Edoxaban and Rivaroxaban also have low aqueous solubility, 0.14 mg/mL and 0.025 mg/mL, respectively.

Anticipated human dose of Apixaban or Razaxaban or Edoxaban or Rivaroxaban or Betrixaban is about 2.5 to 15 mg. To achieve a practical non-oral dose of less than 5 mL, a solution of high drug concentration, for example 2 mg/mL, is required.

Since, the solubility of Apixaban or Razaxaban or Edoxaban or Rivaroxaban could not be increased to needed level i.e. 2 mg/mL, by adjusting pH in physiological range [pH 1.2-6.8] due to pH dependent instability. Hence, a novel co-solvency approach is proposed for current formulation.

### OBJECTIVE OF INVENTION

The objective of the present invention is to provide a novel non-oral formulation of factor Xa inhibitors comprising factor Xa inhibitor and pharmaceutically acceptable excipients selected from vehicles, surface active agents, co-solvents and stabilizers.

Another objective of the present invention is to provide a non-oral formulation of factor Xa inhibitors wherein factor Xa inhibitors are selected from Apixaban, Razaxaban, Edoxaban, Rivaroxaban and Betrixaban.

Another objective of the present invention is to provide a non-oral formulation of factor Xa inhibitors prepared using pharmaceutically acceptable excipients present within permitted IIG limit.

Another objective of the present invention is to provide a process for preparing novel formulation of factor Xa inhibitor for non-oral route of administration.

Another objective of the present invention is to provide a novel non-oral formulation of factor Xa inhibitors for parenteral, topical, nasal, transdermal and ophthalmic administration.

### SUMMARY OF INVENTION

Accordingly, the present invention provides a novel non-oral formulation of factor Xa inhibitors comprising factor Xa inhibitor and pharmaceutically acceptable excipients selected from vehicles, surface active agents, co-solvents and stabilizers.

One embodiment of the present invention provides a non-oral formulation of factor Xa inhibitors with excipients within permitted IIG limit for I.V. administration, wherein factor Xa inhibitors are selected from the group of Apixaban, Razaxaban, Edoxaban, Rivaroxaban and Betrixaban.

Another embodiment of the present invention provides a liquid formulation of factor Xa inhibitors, wherein factor Xa inhibitors are selected from Apixaban, Razaxaban, Edoxaban, Rivaroxaban and Betrixaban.

Another embodiment of the present invention provides a non-oral formulation of factor Xa inhibitors comprising:
a) factor Xa inhibitor,
b) vehicles,
c) surface active agents, and
d) co-solvents.

Another embodiment of the present invention provides a non-oral formulation of factor Xa inhibitors comprising:
a) factor Xa inhibitor,
b) vehicles,
c) surface active agents,
d) stabilizers and
e) co-solvents.

Another embodiment of the present invention provides a non-oral formulation of factor Xa inhibitors comprising:
a) 0.001% to 10% w/w of factor Xa inhibitor,
b) 5% to 90% w/w of vehicles,
c) 0.001% to 40% w/w of surface active agents and
d) 10% to 90% w/w of co-solvents.

Another embodiment of the present invention provides a non-oral formulation of factor Xa inhibitors comprising:
a) 0.001% to 10% w/w of factor Xa inhibitor,
b) 5% to 90% w/w of vehicles,
c) 0.001% to 40% w/w of surface active agents,
d) 10% to 90% w/w of co-solvents, and
e) 1% to 30% w/w of stabilizers.

Another embodiment of the present invention provides a non-oral formulation of factor Xa inhibitors comprising:
a) factor Xa inhibitor selected from Apixaban, Razaxaban, Edoxaban, Rivaroxaban and Betrixaban,
b) water as vehicle,
c) surface active agents selected from polyoxyethylene sorbitan fatty acid esters (Polysorbates), block copolymers of polyethylene glycol and propylene glycol (poloxamers), and
d) co-solvents selected from alcohol, propylene glycol, ethyl alcohol, polyethylene glycol, glycerin, benzyl alcohol, dimethylsulfoxide, dimethylacetamide and combinations thereof.

Another embodiment of the present invention provides a non-oral formulation of factor Xa inhibitors comprising:
a) factor Xa inhibitor selected from Apixaban, Razaxaban, Rivaroxaban and Betrixaban,
Edoxaban,
b) water as vehicle,
c) surface active agents selected from polyoxyethylene sorbitan fatty acid esters (Polysorbates), block copolymers of polyethylene glycol and propylene glycol (poloxamers),
d) co-solvents selected from alcohol, propylene glycol, ethyl alcohol, polyethylene glycol, glycerin, benzyl alcohol, dimethylsulfoxide, dimethylacetamide and combinations thereof and
e) stabilizers selected from glycerin, propylene glycol, sorbitol, and mannitol.

Another embodiment of the present invention provides a non-oral formulation of factor Xa inhibitors comprising:
a) 0.001% to 10% w/w of factor Xa inhibitor selected from Apixaban, Razaxaban, Edoxaban, Rivaroxaban and Betrixaban,
b) 5% to 90% w/w of water as vehicle,
c) 0.001% to 40% w/w of surface active agents selected from polyoxyethylene sorbitan fatty acid esters (Polysorbates), block copolymers of polyethylene glycol and propylene glycol (poloxamers), and
d) 10% to 90% w/w of co-solvents selected from alcohol, propylene glycol, ethyl alcohol, polyethylene glycol, glycerin, benzyl alcohol, dimethylsulfoxide, dimethylacetamide and combinations thereof.

Another embodiment of the present invention provides a non-oral formulation of factor Xa inhibitors comprising:
a) 0.001% to 10% w/w of factor Xa inhibitor selected from Apixaban, Razaxaban, Edoxaban, Rivaroxaban and Betrixaban,
b) 5% to 90% w/w of water as vehicle,
f) 0.001% to 40% w/w of surface active agents selected from polyoxyethylene sorbitan fatty acid esters (Polysorbates), block copolymers of polyethylene glycol and propylene glycol (poloxamers),
g) 10% to 90% w/w of co-solvents selected from alcohol, propylene glycol, ethyl alcohol, polyethylene glycol, glycerin, benzyl alcohol, dimethylsulfoxide, dimethylacetamide and combinations thereof and
c) 1% to 30% w/w of stabilizers selected from glycerin, sorbitol, and mannitol.

Another embodiment of the present invention provides a process for preparing a non-oral formulation of factor Xa inhibitors.

Another embodiment of the present invention provides a process for preparing a non-oral formulation of factor Xa inhibitor for parenteral, topical, nasal, transdermal and ophthalmic administration.

Another embodiment of the present invention provides a process for preparing a non-oral formulation of factor Xa inhibitor comprising:
a) adding factor Xa inhibitor and excipients under continuous mixing using stirring,
b) making the final volume with water for injection, and
c) heating the mixture if required.

Another embodiment of the present invention provides a process for preparing a non-oral formulation of factor Xa inhibitor comprising:
a) dissolving the surface active agent in vehicle,
b) adding one or more co-solvents and factor Xa inhibitor in the vehicle under continuous mixing using stirring,
c) making the final volume with water for injection, and
d) heating the mixture if required.

Another embodiment of the present invention provides a process for preparing a non-oral formulation of factor Xa inhibitor comprising:
a) dissolving the surface active agent in vehicle,
b) adding one or more co-solvents, stabilizers and factor Xa inhibitor in the vehicle under continuous mixing using stirring,
c) making the final volume with water for injection, and
d) heating the mixture if required.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The solubility of Apixaban, Razaxaban, Edoxaban, Rivaroxaban and Betrixaban in this vehicle is at least 1.5 mg/mL. The present invention has better acceptability as compared to earlier claimed inventions of the same group of drugs.

In an embodiment of present invention, the formulation is suitable for bolus injection, intravenous infusion, topical, nasal, transdermal and ophthalmic administration.

In another embodiment of the present invention is a method for treating the risk of stroke and systemic embolism in nonvalular atrial fibrillation patients and deep vein thrombosis and pulmonary embolism after hip or knee replacement surgery, administered to patient in need, thereof a non-oral formulation of Apixaban, Razaxaban, Edoxaban, Rivaroxaban and Betrixaban are therapeutically active.

The subject invention describes new formulations of factor Xa inhibitor such as Apixaban, Razaxaban, Edoxaban, Rivaroxaban and Betrixaban for administration which includes non-oral formulation and method for making and using the same.

Drug solubility is minimum requirement for designing any dosage form and it is more critical when designing a formulation of poorly soluble drug like factor Xa inhibitor such as Apixaban, Razaxaban, Edoxaban, Rivaroxaban and Betrixaban. Because of low solubility associated with factor Xa inhibitor such as Apixaban, Razaxaban, Edoxaban, Rivaroxaban and Betrixaban, the bioavailability is limited and hence, solubility enhancement is necessary.

The method of making and using novel non-oral formulation of poorly soluble factor Xa inhibitor such as Apixaban, Razaxaban, Edoxaban, Rivaroxaban and Betrixaban disclosed in this invention addresses the enhancement of patient acceptability for same class of drugs.

In accordance with current invention, it was observed that water solubility of factor Xa inhibitor was sufficiently increased to allow preparation of aqueous based non-oral formulation by using factor Xa inhibitor with surface active agents and co-solvents and solvent and solubilising agents and stabilizers.

Many different category of water miscible co-solvent system and solubilizing agents have proved to be unacceptable for liquid formulation of factor Xa inhibitor due to the limited solubility of Apixaban, Razaxaban, Edoxaban, Rivaroxaban and Betrixaban sufficiently to provide a drug concentration of at least 1.5 mg/mL at acceptable administrable volume.

The factor Xa inhibitor is selected from Apixaban, Razaxaban, Edoxaban, Rivaroxaban and Betrixaban.

The factor Xa inhibitor used in formulations of present invention is in the range of about 0.001% to 10% w/w of total weight of the formulation.

The vehicle used in the formulations of the present invention is water.

The vehicle used in formulations of present invention is in the range of about 5% to 90% w/w of total weight of the formulation.

The surface active agents used in the present invention are selected from non-ionic surface active agents such as polyoxyethylene sorbitan fatty acid esters (Polysorbates), block copolymers of polyethylene glycol and propylene glycol (poloxamers), polyoxyethylene castor oil derivative, polyoxyglyceride, vitamin E polyethylene glycol succinate, and macrogol 15 hydroxystearate and combinations thereof.

The surface active agents used in formulations of present invention is in the range of about 0.001% to 40% w/w of total weight of the formulation.

The co-solvents used in the present invention are selected from alcohol, propylene glycol, ethyl alcohol, polyethylene glycol, glycerin, benzyl alcohol, ester of a medium or long chain fatty acid such as a mono- di- or triglyceride, phospholipids, dimethylsulfoxide, dimethylacetamide and combinations thereof.

The co-solvents used in formulations of present invention is in the range of about 1% to 90% w/w of total weight of the formulation.

The stabilizers used in the present invention are selected from glycerin, sorbitol, and mannitol.

The stabilizers used in formulations of present invention are in the range of about 1% to 90% w/w of total weight of the formulation.

The said invention is stable as confirmed from stability studies conducted on prepared formulations.

The product thus formulated can be used for treatment of risk of stroke, systemic embolism in patients with nonvalvular atrial fibrillation, deep vein thrombosis and pulmonary embolism after hip or knee replacement surgery. As with any potent drug, the dosage must be individualized by treating clinician.

The formulation of the present invention is manufactured by dissolving surface active agent in vehicle followed by adding one or more co-solvent's or co-solvent and stabilizer and finally adding factor Xa inhibitor in the vehicle under continuous mixing using stirring. Final volume was made up by water for injection. Mixture was heated at 40-80 °C for 10-30 minutes if required, to obtain clear solution upon cooling to room temperature.

The solution was free from particulate matter by visual inspection and was filtered by sterilizing grade filter followed by aseptic filling into vials or ampoules or prefilled syringes. If required, the final product was sterilized by autoclaving. Target fill volume was 3.5 mL including 0.5 mL overfill for vial or needle or ampoule hold up.

The following examples describe the nature of the invention and are given only for the purpose of illustrating the present invention in more detail and are not limitative.

### Example 1

TABLE NO 1
Composition for formulation 1

| | | | | Amount per |
|---|---|---|---|---|
| | | Amount | Amount | dosage unit |
| Ingredient | Function | (mg/mL) | (μL/mL) | (mg) |
| Apixaban | Active | 1.5 | — | 4.5 |
| Water | Vehicle | 400 | 400 | 1200 |
| Polysorbate 80 | Surface active | 5 | 5 | 15 |
| | agent | | | |
| Propylene glycol | Co-Solvent | 230 | 221 | 690 |
| Alcohol | Co-Solvent | 300 | 380 | 900 |

### Manufacturing Process

Dissolving Polysorbate 80 in water followed by adding Propylene glycol, Alcohol and finally adding Apixaban in the vehicle. Each component was added under continuous mixing using stirring. Final volume was made up by water for injection. Mixture was heated at 55-60 °C for 10-30 minutes if required, to obtain clear solution upon cooling to room temperature. The solution was free from particulate matter by visual inspection and was filtered by sterilizing grade filter followed by aseptic filling into vials or ampoules or prefilled syringes. If required, the final product was sterilized by autoclaving. Target fill volume was 3.5 mL including 0.5 mL overfill for vial or needle or ampoules hold up.

### Example 2

TABLE NO 2
Composition for formulation 2

| | | | | Amount per |
|---|---|---|---|---|
| | | Amount | Amount | dosage unit |
| Ingredient | Function | (mg/mL) | (μL/mL) | (mg) |
| Apixaban | Active | 2.0 | — | 6.0 |
| Water | Vehicle | 70 | 70 | 210 |
| Polysorbate 80 | Surface active | 40 | 37 | 120 |
| | agent | | | |
| Propylene glycol | Co-Solvent | 400 | 385 | 1200 |
| Alcohol | Co-Solvent | 400 | 506 | 1200 |

The formulations of example 2 were prepared as per manufacturing process described in Example 1.

### Example 3

TABLE NO 3
Composition for formulation 3

| | | | | Amount per |
|---|---|---|---|---|
| | | Amount | Amount | dosage unit |
| Ingredient | Function | (mg/mL) | (μL/mL) | (mg) |
| Apixaban | Active | 1.5 | — | 4.5 |
| Water | Vehicle | 400 | 400 | 1200 |
| Poloxamer 188 | Surface active | 6 | — | 18 |
| | agent | | | |
| Propylene glycol | Co-Solvent | 230 | 221 | 690 |
| Alcohol | Co-Solvent | 300 | 380 | 900 |

The formulations of example 3 were prepared as per manufacturing process described in Example 1.

### Example 4

TABLE NO 4
Composition for formulation 4

| | | | | Amount per |
|---|---|---|---|---|
| | | Amount | Amount | dosage unit |
| Ingredient | Function | (mg/mL) | (μL/mL) | (mg) |
| Apixaban | Active | 1.7 | — | 5.1 |
| Water | Vehicle | 345 | 345 | 1035 |
| Poloxamer 188 | Surface active | 224 | — | 672 |
| | agent | | | |
| Propylene glycol | Co-Solvent | 200 | 193 | 600 |
| Alcohol | Co-Solvent | 200 | 253 | 600 |

The formulations of example 4 were prepared as per manufacturing process described in Example 1.

### Example 5

TABLE NO 5
Composition for formulation 5

| | | | | Amount per |
|---|---|---|---|---|
| | | Amount | Amount | dosage unit |
| Ingredient | Function | (mg/mL) | (μL/mL) | (mg) |
| Apixaban | Active | 1.2 | — | 3.6 |
| Water | Vehicle | 550 | 550 | 1650 |
| Polysorbate 80 | Surface active | 5 | 5 | 15 |
| | agent | | | |
| Sorbitol | Stabilizer | 100 | 67 | 300 |
| Alcohol | Co-Solvent | 300 | 380 | 900 |

The formulations of example 5 were prepared as per manufacturing process described in Example 1.

### Example 6

TABLE NO 6
Composition for formulation 6

| | | | | Amount per |
|---|---|---|---|---|
| | | Amount | Amount | dosage unit |
| Ingredient | Function | (mg/mL) | (μL/mL) | (mg) |
| Apixaban | Active | 1.6 | — | 4.8 |
| Water | Vehicle | 450 | 450 | 1350 |
| Polysorbate 80 | Surface active | 40 | 38 | 120 |
| | agent | | | |
| Sorbitol | Stabilizer | 300 | 200 | 900 |
| Alcohol | Co-Solvent | 250 | 320 | 750 |

The formulations of example 6 were prepared as per manufacturing process described in Example 1.

### Example 7

TABLE NO 7
Composition for formulation 7

| | | | | Amount per |
|---|---|---|---|---|
| | | Amount | Amount | dosage unit |
| Ingredient | Function | (mg/mL) | (μL/mL) | (mg) |
| Razaxaban | Active | 1.0 | — | 3.0 |
| Water | Vehicle | 550 | 550 | 1650 |
| Polysorbate 80 | Surface active | 5 | 5 | 15 |
| | agent | | | |
| Mannitol | Stabilizer | 100 | — | 300 |
| Alcohol | Co-Solvent | 300 | 380 | 900 |

The formulations of example 7 were prepared as per manufacturing process described in Example 1.

### Example 8

TABLE NO 8
Composition for formulation 8

| | | | | Amount per |
|---|---|---|---|---|
| | | Amount | Amount | dosage unit |
| Ingredient | Function | (mg/mL) | (μL/mL) | (mg) |
| Apixaban | Active | 1.2 | — | 3.6 |
| Water | Vehicle | 450 | 450 | 1350 |
| Polysorbate 80 | Surface active | 40 | 40 | 120 |
| | agent | | | |
| Mannitol | Stabilizer | 300 | — | 900 |
| Alcohol | Co-Solvent | 250 | 320 | 750 |

The formulations of example 8 were prepared as per manufacturing process described in Example 1.

### Example 9

TABLE NO 9
Composition for formulation 9

| | | | | Amount per |
|---|---|---|---|---|
| | | Amount | Amount | dosage unit |
| Ingredient | Function | (mg/mL) | (μL/mL) | (mg) |
| Edoxaban | Active | 1.3 | — | 3.9 |
| Water | Vehicle | 550 | 550 | 1650 |
| Poloxamer 188 | Surface active | 6 | — | 18 |
| | agent | | | |
| Sorbitol | Stabilizer | 100 | 67 | 300 |
| Alcohol | Co-Solvent | 300 | 380 | 900 |

The formulations of example 9 were prepared as per manufacturing process described in Example 1.

### Example 10

TABLE NO 10
Composition for formulation 10

| | | | | Amount per |
|---|---|---|---|---|
| | | Amount | Amount | dosage unit |
| Ingredient | Function | (mg/mL) | (μL/mL) | (mg) |
| Rivaroxaban | Active | 1.2 | — | 3.6 |
| Water | Vehicle | 345 | 345 | 1035 |
| Poloxamer 188 | Surface active | 224 | — | 672 |
| | agent | | | |
| Sorbitol | Stabilizer | 300 | 200 | 900 |
| Alcohol | Co-Solvent | 200 | 253 | 600 |

The formulations of example 10 were prepared as per manufacturing process described in Example 1.

### Example 11

TABLE NO 11
Composition for formulation 11

| | | | | Amount per |
|---|---|---|---|---|
| | | Amount | Amount | dosage unit |
| Ingredient | Function | (mg/mL) | (μL/ml) | (mg) |
| Apixaban | Active | 1.0 | — | 3.0 |
| Water | Vehicle | 550 | 550 | 1650 |
| Poloxamer 188 | Surface active agent | 6 | — | 18 |
| Mannitol | Stabilizer | 100 | — | 300 |
| Alcohol | Co-Solvent | 300 | 380 | 900 |

The formulations of example 11 were prepared as per manufacturing process described in Example 1.

### Example 12

TABLE NO 12
Composition for formulation 12

| | | | | Amount per |
|---|---|---|---|---|
| | | Amount | Amount | dosage unit |
| Ingredient | Function | (mg/mL) | (μL/mL) | (mg) |
| Apixaban | Active | 1.2 | — | 3.6 |
| Water | Vehicle | 345 | 345 | 1035 |
| Poloxamer 188 | Surface active | 224 | — | 672 |
| | agent | | | |
| Mannitol | Stabilizer | 300 | — | 900 |
| Alcohol | Co-Solvent | 200 | 253 | 600 |

The formulations of example 12 were prepared as per manufacturing process described in Example 1.

### Example 13

TABLE NO 13
Composition for formulation 13

| | | | | Amount per |
|---|---|---|---|---|
| | | Amount | Amount | dosage unit |
| Ingredient | Function | (mg/mL) | (μL/mL) | (mg) |
| Apixaban | Active | 1.4 | — | 4.2 |
| Water | Vehicle | 440 | 440 | 1320 |
| Polysorbate 80 | Surface active | 5 | 5 | 15 |
| | agent | | | |
| Glycerine | Stabilizer | 230 | 182 | 690 |
| Alcohol | Co-Solvent | 300 | 380 | 900 |

The formulations of example 13 were prepared as per manufacturing process described in Example 1.

### Example 14

TABLE NO 14
Composition for formulation 14

| | | | | Amount per |
|---|---|---|---|---|
| | | Amount | Amount | dosage unit |
| Ingredient | Function | (mg/mL) | (μL/mL) | (mg) |
| Apixaban | Active | 1.7 | — | 5.1 |
| Water | Vehicle | 140 | 140 | 420 |
| Polysorbate 80 | Surface active | 40 | 38 | 120 |
| | agent | | | |
| Glycerine | Stabilizer | 400 | 317 | 1200 |
| Alcohol | Co-Solvent | 400 | 506 | 1200 |

The formulations of example 14 were prepared as per manufacturing process described in Example 1.

### Example 15

TABLE NO 15
Composition for formulation 15

| | | | | Amount per |
|---|---|---|---|---|
| | | Amount | Amount | dosage unit |
| Ingredient | Function | (mg/mL) | (μL/mL) | (mg) |
| Apixaban | Active | 1.0 | — | 3.0 |
| Water | Vehicle | 500 | 500 | 1500 |
| Polysorbate 80 | Surface active | 5 | 5 | 15 |
| | agent | | | |
| Propylene glycol | Co-Solvent | 230 | 221 | 690 |
| Benzyl alcohol | Co-Solvent | 300 | 288 | 900 |

The formulations of example 15 were prepared as per manufacturing process described in Example 1.

### Example 16

TABLE NO 16
Composition for formulation 16

| | | | | Amount per |
|---|---|---|---|---|
| | | Amount | Amount | dosage unit |
| Ingredient | Function | (mg/mL) | (μL/mL) | (mg) |
| Apixaban | Active | 1.2 | — | 3.6 |
| Water | Vehicle | 200 | 200 | 600 |
| Polysorbate 80 | Surface active | 40 | 38 | 120 |
| | agent | | | |
| Propylene glycol | Co-Solvent | 400 | 385 | 1200 |
| Benzyl alcohol | Co-Solvent | 400 | 385 | 1200 |

The formulations of example 16 were prepared as per manufacturing process described in Example 1.

### Example 17

TABLE NO 17
Composition for formulation 17

| | | | | Amount per |
|---|---|---|---|---|
| | | Amount | Amount | dosage unit |
| Ingredient | Function | (mg/mL) | (μL/mL) | (mg) |
| Apixaban | Active | 1.0 | — | 3.0 |
| Water | Vehicle | 700 | 700 | 2100 |
| Polysorbate 80 | Surface active | 5 | 5 | 15 |
| | agent | | | |
| Propylene glycol | Co-Solvent | 230 | 221 | 690 |
| Dimethyl | Co-Solvent | 100 | 91 | 300 |
| Sulfoxide | | | | |

The formulations of example 17 were prepared as per manufacturing process described in Example 1.

### Example 18

TABLE NO 18
Composition for formulation 18

| | | | | Amount per |
|---|---|---|---|---|
| | | Amount | Amount | dosage unit |
| Ingredient | Function | (mg/mL) | (μL/mL) | (mg) |
| Apixaban | Active | 1.2 | — | 3.6 |
| Water | Vehicle | 500 | 500 | 1500 |
| Polysorbate 80 | Surface active | 40 | 38 | 120 |
| | agent | | | |
| Propylene glycol | Co-Solvent | 400 | 385 | 1200 |
| Dimethyl | Co-Solvent | 100 | 91 | 300 |
| Sulfoxide | | | | |

The formulations of example 18 were prepared as per manufacturing process described in Example 1.

### Example 19

TABLE NO 19
Composition for formulation 19

| | | | | Amount per |
|---|---|---|---|---|
| | | Amount | Amount | dosage unit |
| Ingredient | Function | (mg/mL) | (μL/mL) | (mg) |
| Apixaban | Active | 1.0 | — | 3.0 |
| Water | Vehicle | 570 | 570 | 1710 |
| Polysorbate 80 | Surface | 5 | 5 | 15 |
| | active agent | | | |
| Propylene glycol | Co-Solvent | 230 | 221 | 690 |
| Dimethylacetamide | Co-Solvent | 200 | 210 | 600 |

The formulations of example 19 were prepared as per manufacturing process described in Example 1.

### Example 20

TABLE NO 20
Composition for formulation 20

| | | | | Amount per |
|---|---|---|---|---|
| | | Amount | Amount | dosage unit |
| Ingredient | Function | (mg/mL) | (μL/mL) | (mg) |
| Apixaban | Active | 1.2 | — | 3.6 |
| Water | Vehicle | 260 | 260 | 780 |
| Polysorbate 80 | Surface | 40 | 38 | 120 |
| | active agent | | | |
| Propylene glycol | Co-Solvent | 400 | 385 | 1200 |
| Dimethylacetamide | Co-Solvent | 300 | 320 | 900 |

The formulations of example 20 were prepared as per manufacturing process described in Example 1.

While the invention has been described with reference to a particular embodiment, it will be understood by those expert in the skill that various alterations may be made without deviating from the scope of invention. In addition to this, several alterations may be made to acclimatize a particular invention without deviating from the major scope. It is believed that the invention must not be restricted to particular embodiment disclosed as the best mode anticipated for carrying out of this invention, but then the invention will include all.

1. A novel non-oral formulation of factor Xa inhibitors comprising factor Xa inhibitor and pharmaceutically acceptable excipients selected from vehicles, surface active agents, co-solvents and stabilizers.

2. The formulation as claimed in claim 1, wherein factor Xa inhibitor is selected from the group of Apixaban, Razaxaban, Edoxaban, Rivaroxaban and Betrixaban.

3. The formulation as claimed in claim 1, wherein the said factor Xa inhibitor has solubility from 1.5 to about 2 mg/mL.

4. The formulation as claimed in claim 1, wherein vehicle is water.

5. The formulation as claimed in claim 1, wherein non-ionic surface active agents selected from polyoxyethylene sorbitan fatty acid esters (Polysorbates), block copolymers of polyethylene glycol and propylene glycol (poloxamers), polyoxyethylene castor oil derivative, polyoxyglyceride, vitamin E polyethylene glycol succinate, and macrogol 15 hydroxystearate and combinations thereof.

6. The formulation as claimed in claim 1, wherein co-solvents selected from alcohol, propylene glycol, ethyl alcohol, polyethylene glycol, glycerin, benzyl alcohol, ester of a medium or long chain fatty acid such as a mono- di- or triglyceride, phospholipids, dimethylsulfoxide, dimethylacetamide and combinations thereof.

7. The formulation as claimed in claim 1, wherein stabilizers are selected from glycerin, sorbitol, and mannitol.

8. The formulation as claimed in claim 1, wherein non-oral formulation of factor Xa inhibitors comprising:
a) 0.001% to 5% w/w of factor Xa inhibitor selected from Apixaban, Razaxaban, Edoxaban, Rivaroxaban and Betrixaban,
b) 5% to 90% w/w of water as vehicle,
c) 0.001% to 40% w/w of surface active agents selected from polyoxyethylene sorbitan fatty acid esters (Polysorbates), block copolymers of polyethylene glycol and propylene glycol (poloxamers), and
d) 10% to 90% w/w of co-solvents selected from alcohol, propylene glycol, ethyl alcohol, polyethylene glycol, glycerin, benzyl alcohol, dimethylsulfoxide, dimethylacetamide and combinations thereof.

9. The formulation as claimed in claim 1, wherein non-oral formulation of factor Xa inhibitors comprising:
a) 0.001% to 5% w/w of factor Xa inhibitor selected from Apixaban, Razaxaban, Edoxaban, Rivaroxaban and Betrixaban,
b) 5% to 90% w/w of water as vehicle,
c) 0.001% to 40% w/w of surface active agents selected from polyoxyethylene sorbitan fatty acid esters (Polysorbates), block copolymers of polyethylene glycol and propylene glycol (poloxamers),
d) 10% to 90% w/w of co-solvents selected from alcohol, propylene glycol, ethyl alcohol, polyethylene glycol, glycerin, benzyl alcohol, dimethylsulfoxide, dimethylacetamide and combinations thereof and
e) 1% to 30% w/w of stabilizers selected from glycerin, sorbitol, and mannitol.

10. The process for the preparation of formulation as claimed in claim 1, wherein said a process comprising:
a) dissolving the surface active agent in vehicle,
b) adding one or more co-solvents and factor Xa inhibitor in the vehicle under continuous mixing using stirring,
c) making the final volume with water for injection, and
d) heating the mixture.

11. The process for the preparation of formulation as claimed in claim 1, wherein said a process comprising:
a) dissolving the surface active agent in vehicle,
b) adding one or more co-solvents, stabilizers and factor Xa inhibitor in the vehicle under continuous mixing using stirring,
c) making the final volume with water for injection, and
d) heating the mixture.
